(19) United States
(12) Patent Application Publication
(10) Pub. No.: US 2017/0088482 A1
(43) Pub. Date: Mar. 30, 2017

(54) Process and plant for producing olefins

(71) Applicant: Linde Aktiengesellschaft, München (DE)

(72) Inventors: Torben Höfel, München (DE); Harald Schmaderer, Wolfratshausen (DE); Ernst Haidegger, Riemerling (DE); Clara Delhomme-Neudecker, München (DE); Helmut Fritz, München (DE); Marianne Ponceau, München (DE)

(21) Appl. No.: 15/117,044

(22) PCT Filed: Feb. 6, 2015
(86) PCT No.: PCT/EP2015/052547

§ 371 Date: Aug. 5, 2016

(30) Foreign Application Priority Data
Feb. 7, 2014 (DE) 10 2014 202 285.1

(51) Int. Cl. C07C 1/20; C07C 4/04

(52) U.S. Cl. CPC C07C 1/20; C07C 4/04

(57) ABSTRACT

A method (100) for producing olefins is proposed, wherein a first gas mixture (b) is produced by means of a steam cracking process (1) and a second gas mixture (s) is produced by means of an oxygenate-to-olefin process (2), the first gas mixture (b) and the second gas mixture (s) each containing at least hydrocarbons with one to four carbon atoms. It is proposed that from the first gas mixture (b) a first fraction (h) is formed which contains at least the great majority of the hydrocarbons with four carbon atoms previously contained in the first gas mixture (b), from the second gas mixture (s) a second fraction (y) is formed which contains at least the great majority of the hydrocarbons with four carbon atoms previously contained in the second gas mixture (s), and that the hydrocarbons contained in the second fraction (y) and previously in the second gas mixture (s) are predominantly subjected in the steam cracking process (1) to cracking conditions under which any n-butane present is reacted by less than 92%. The invention further relates to an apparatus for this purpose.

The invention relates to a method and an apparatus for producing olefins according to the pre-characterising clauses of the independent claims.

### PRIOR ART

Short-chain olefins such as ethylene and propylene can be produced by steam-cracking hydrocarbons, as explained in detail hereinafter. Alternative methods of obtaining short-chain olefins of this kind are the so-called oxygenate-to-olefin methods (in English: Oxygenates to Olefins, OTO).

By oxygenates are meant oxygen-containing compounds derived from saturated hydrocarbons, particularly ethers and alcohols. Oxygenates are used for example as fuel additives for increasing the octane number and as a lead substitute (cf. D. Barceló (ed.): Fuel Oxygenates, in D. Barceló and A. G. Kostianoy (ed.): The Handbook of Environmental Chemistry, vol. 5, Heidelberg: Springer, 2007). The addition of oxygenates to fuels leads, among other things, to cleaner burning in the engine and thereby reduces emissions.

Corresponding oxygenates are typically ethers and alcohols. Besides methyl tert. butyl ether (MTBE), it is also possible to use, for example, tert. amyl methyl ether (TAME), tert. amyl ethyl ether (TAEE), ethyl tert. butyl ether (ETBE) and diisopropyl ether (DIPE). Alcohols which may be used include for example methanol, ethanol and tert. butanol (TBA). The oxygenates also include, in particular, the dimethyl ether described hereinafter (DME, dimethyl ether). The invention is not limited to the fuel additives mentioned but is equally suitable for use with other oxygenates.

According to a common definition which is also used here, oxygenates are compounds which comprise at least one alkyl group covalently bonded to an oxygen atom. The at least one alkyl group may comprise up to five, up to four or up to three carbon atoms. In particular, the oxygenates which are of interest within the scope of the present invention, comprise alkyl groups with one or two carbon atoms, particularly methyl groups. In particular they are monohydric alcohols and dialkyl ethers such as methanol and dimethyl ether or corresponding mixtures thereof.

In oxygenate-to-olefin methods, corresponding oxygenates such as methanol or dimethyl ether are introduced into a reaction zone of a reactor in which a catalyst suitable for reacting the oxygenates is provided. The catalyst typically contains a molecular sieve. Under the effect of the catalyst the oxygenates are converted into ethylene and propylene, for example. The catalysts and reaction conditions used in oxygenate-to-olefin methods are basically known to the skilled man.

The invention may operate with different catalysts in the oxygenate-to-olefin method. For example, zeolites such as ZSM-5 or SAPO-34 or functionally comparable materials may be used. If ZSM-5 or a comparable material is used, comparatively large amounts of longer-chained (C3plus) hydrocarbons and comparatively small amounts of shorter-chained (C2minus) hydrocarbons are formed. When SAPO-34 or comparable materials are used, by contrast, significant amounts of shorter-chained (C2minus) hydrocarbons are also formed.

Special forms of oxygenate-to-olefin processes are present if corresponding reactors are charged not with the oxygenates that actually give their name to the oxygenate-to-olefin methods but with olefins. From a technical point of view and in their procedures, such processes differ only slightly or not at all from the oxygenate-to-olefin methods in the narrower sense, apart from the components used (cf. J. C. Bricker et al: New Catalytic Technologies for the Industrial Production of Ethylene and Propylene in: Science and Technology in Catalysis 2006, Amsterdam: Elsevier, 2006).

Therefore, by an oxygenate-to-olefin method is meant, hereinafter, both a method in which one or more of the above-mentioned oxygenates (not only methanol and/or dimethyl ether) are at least partially reacted by catalytic conversion to form olefins, but also a method in which a corresponding reactor is charged with a predominantly olefinic feed. It is also possible to use a plurality of reactors charged with different feeds and/or operated under different conditions and/or with different catalysts.

Integrated methods and apparatus (combined apparatus) for producing hydrocarbons which comprise steam cracking processes and oxygenate-to-olefin methods or corresponding cracking furnaces and reactors are known and are described for example in WO 2011/057975 A2 or US 2013/172627 A1.

Integrated methods of this kind are advantageous because typically not only the desired short-chain olefins are formed in the oxygenate-to-olefin processes. A substantial proportion of the oxygenates is converted into paraffins and C4plus olefins (for the designations see below). At the same time, in steam cracking, the entire furnace feed is not cracked into short-chain olefins. As yet unreacted paraffins may be present in the cracked gas of corresponding cracking furnaces. Moreover, C4plus olefins including diolefins such as butadiene are typically found here. The compounds obtained depend in both cases on the feeds and reaction conditions used.

In the methods proposed in WO2011/057975 A2 and US 2013/172627 A1 the cracked gas of a cracking furnace and the offstream from an oxygenate-to-olefin reactor are combined in a joint separating unit and fractionated. A C4 fraction may be subjected to a further steam cracking and/or oxygenate-to-olefin process, for example after hydrogenation or separation of butadiene. The C4 fraction may be separated into predominantly olefinic and predominantly paraffinic partial fractions.

The utilisation of the compounds contained in this C4 fraction and the formation of the desired target compounds, however, does not always prove satisfactory in the methods described. In addition, the separation into predominantly olefinic and predominantly paraffinic partial fractions is extremely laborious.

### DISCLOSURE OF THE INVENTION

Against this background the present invention proposes a method and an apparatus for producing olefins having the features of the independent claims. Preferred embodiments are the subject of the dependent claims and the description that follows.

Before the explanation of the features and advantages of the present invention, their basis and the terminology used will be explained.

The abbreviations used within the scope of this application in the conventional manner for hydrocarbon mixtures or hydrocarbon fractions are based on the carbon number of the compounds that are predominantly or exclusively obtained. Thus, a C1 fraction is a fraction which predominantly or exclusively contains methane (but by convention also contains hydrogen in some cases, and is then also called a “C1minus fraction”). A C2 fraction on the other hand predominantly or exclusively contains ethane, ethylene and/or acetylene. A C3 fraction predominantly contains propane, propylene, methyl acetylene and/or propadiene. A C4 fraction predominantly or exclusively contains butane, butene, butadiene and/or butyne, while the respective isomers may be present in different amounts depending on the source of the C4 fraction. The same also applies to a C5 fraction and the higher fractions. Several such fractions may also be combined in one process and/or under one heading. For example, a C2plus fraction predominantly or exclusively contains hydrocarbons with two or more carbon atoms and a C2minus fraction predominantly or exclusively contains hydrocarbons with one or two carbon atoms and hydrogen.

Methods and apparatus for steam cracking hydrocarbons are known and are described for example in the article “Ethylene” in Ullmann's Encyclopedia of Industrial Chemistry, online since 15 Apr. 2007, DOI 10.1002/14356007.a10_045.pub2.

Steam cracking processes are carried out on a commercial scale predominantly in tubular reactors in which the reaction tubes, the so-called coils, may be operated individually or in groups under identical or different cracking conditions. Reaction tubes or sets of reaction tubes operated under identical or comparable cracking conditions and possibly also tube reactors operated under uniform cracking conditions are each referred to hereinafter as “cracking furnaces”. A cracking furnace, in the terminology used here, is thus a construction unit used for steam cracking which exposes a furnace feed to identical or comparable cracking conditions. A steam cracking apparatus may comprise one or more cracking furnaces.

A whole furnace may possibly be subdivided into two or more cracking furnaces. These are then often referred to as furnace cells. A plurality of furnace cells belonging to one whole furnace generally have radiation zones that are independent of one another and a common convection zone as well as a common smoke exhaust. In these cases, each furnace cell may be operated with its own cracking conditions. Each furnace cell is thus a construction unit used for steam cracking which exposes a furnace feed to identical or comparable cracking conditions and is consequently referred to herein as a cracking furnace. The furnace as a whole then comprises a plurality of corresponding units or, to put it differently, a plurality of cracking furnaces. If there is only one furnace cell, this is the cracking unit and hence the cracking furnace. Cracking furnaces may be combined in groups which are supplied with the same furnace feed, for example. The cracking conditions within a furnace group are generally set to be the same or similar.

In more recent methods and apparatus for steam cracking, mild cracking conditions are increasingly used (see below), as they are able to produce, in particular, so-called high value products such as propylene or butadiene in comparatively large amounts.

The term “furnace feed” here denotes one or more liquid and/or gaseous streams which are supplied to one or more cracking furnaces. Streams obtained from a corresponding steam cracking process, as explained hereinafter, may also be recycled into one or more cracking furnaces and used again as a furnace feed. Suitable furnace feeds include a number of hydrocarbons and hydrocarbon mixtures from ethane to gas oil up to a boiling point of typically 600° C.

A furnace feed may consist of a so-called “fresh feed”, i.e. a feed which is prepared outside the apparatus and is obtained for example from one or more petroleum fractions, petroleum gas and/or petroleum gas condensates. A furnace feed may also consist of one or more so-called “recycle streams”, i.e. streams that are produced in the apparatus itself and recycled into a corresponding cracking furnace. A furnace feed may also consist of a mixture of one or more fresh feeds with one or more recycle streams.

The furnace feed is at least partly reacted in the cracking furnace and leaves it as a so-called “crude gas” which can be subjected to after-treatment steps. These encompass, first of all, processing of the crude gas, for example by quenching, compressing, liquefying, cooling and drying, so as to obtain a so-called “cracked gas”. Occasionally the crude gas is also referred to as cracking gas.

The “cracking conditions” in a cracking furnace mentioned above encompass inter alia the partial pressure of the furnace feed, which may be influenced by the addition of different amounts of steam and the pressure selected in the cracking furnace, the dwell time in the cracking furnace and the temperatures and temperature profiles used therein. The furnace geometry and configuration also play a part. To produce ethylene, a cracking furnace is typically operated at a furnace entry temperature of 500 to 680° C. and at a furnace exit temperature of 775 to 875° C. The “furnace exit temperature” is the temperature of a gas stream at the end of one or more reaction tubes. Typically, this is the maximum temperature to which the gas stream in question is heated. The pressure used, also measured at the end of one or more reaction tubes, is typically 165 to 225 kPa. Steam is mixed with the furnace feed in a ratio of typically 0.25 to 0.85 kg of steam per kg of dry feed. The values used are dependent on the furnace feed used and the desired cracking products.

As the values mentioned influence one another at least partially, the term “cracking severity” has been adopted to characterise the cracking conditions. For liquid furnace feeds, i.e. longer-chain hydrocarbons, the cracking severity can be described by means of the ratio of propylene to ethylene (P/E) or as the ratio of methane to propylene (M/P) in the cracked gas, based on weight (kg/kg). The P/E and M/P ratios are directly dependent on the temperature, but, unlike the real temperature in or at the exit from a cracking furnace, they can be measured more accurately and be used for example as a control variable in a regulating process.

For gaseous furnace feeds the reaction or conversion of a particular component of the furnace feed may be specified as a measure of the cracking severity. In particular, for the C4 fractions or C4 partial streams used in the present case, it is useful, and conventional in the art, to describe the cracking severity in terms of the reaction of key components such as n-butane.

The cracking severities or cracking conditions are “severe” if n-butane in a corresponding fraction is reacted by more than 92%. Under even more severe cracking conditions, n-butane is optionally reacted by more than 93%, 94% or 95%. Typically, there is no 100% reaction of n-butane. The upper limit of the “severe” cracking severities or cracking conditions is therefore 99%, 98%, 97% or 96% reaction of n-butane, for example. The cracking severities or cracking conditions are “mild”, on the other hand, if n-butane is reacted by less than 92%. At less than 91%, less than 90%, less than 89%, less than 88% or less than 87% reaction of n-butane, ever milder cracking severities or cracking conditions are present. At less than 86% reaction of n-butane the cracking severities or cracking conditions are referred to as “very mild”. Very mild cracking severities or cracking conditions also encompass, for example, a reaction of n-butane of less than 85%, 80% or 70% and more than 50% or 60%.

The above-mentioned cracking severities or cracking conditions are correlated in particular with the furnace exit temperature at the end of the reaction tube or tubes or the cracking furnaces used, as described above. The higher this temperature, the more “severe”, and the lower the temperature, the “milder” the cracking severities or cracking conditions.

It should also be understood that the reaction of other components does not have to be identical to that of n-butane. A percentage reaction of a key component, in this case n-butane, is however associated with a furnace exit temperature and the respective percentage reactions of the other components in the feedstock. This furnace exit temperature is in turn dependent on the cracking furnace, among other things. The difference between the respective percentage reactions is dependent on a number of other factors.

### Advantages of the Invention

The present invention starts from a known method of obtaining olefins in which a first gas mixture is obtained by a steam cracking process and a second gas mixture is produced by an oxygenate-to-olefin process, the first gas mixture and the second gas mixture each containing at least hydrocarbons with one to four carbon atoms. The first and/or second gas mixture may also contain hydrocarbons with more than four carbon atoms and/or hydrogen, as well as other compounds, including components which have not reacted in the steam cracking process and/or the oxygenate-to-olefin process. However, within the scope of the present invention, the first and second gas mixtures are not completely subjected to a joint separation process as known from the above-mentioned WO 2011/057975 A2 and/or US 2013/172627 A1, for example.

Rather, the invention provides that a first fraction is formed from the first gas mixture (which is produced by the steam cracking method) which contains at least the great majority of the hydrocarbons with four carbon atoms previously contained in the first gas mixture, and a second fraction is formed from the second gas mixture (which is produced by the oxygenate-to-olefin process) which contains at least the great majority of the hydrocarbons with four carbon atoms previously contained in the second gas mixture. The separation of the two gas mixtures is thus carried out at least partially separately from one another, which has the advantage that, with first and second gas mixtures of different compositions, the products obtained from them can be treated separately in a controlled manner.

By the “great majority” is meant, within the scope of the present application, a proportion of at least 75%, 80%, 85%, 90%, 99% or more.

Within the scope of the present invention the C4 and optionally longer-chained hydrocarbons from the second gas mixture may be recycled into the steam cracking process without any further treatment, i.e. separation or chemical reaction, and there they are subjected to mild cracking conditions.

It has been found according to the invention that in the steam cracking process the hydrocarbons contained in the second fraction and previously in the second gas mixture can particularly advantageously be predominantly subjected to cracking conditions in which n-butane contained in the second fraction is reacted by less than 92%.

In other words, the hydrocarbons with four or optionally more carbon atoms contained in the second gas mixture, i.e. in a product stream of an oxygenate-to-olefin process, are preferably cracked under mild cracking conditions, but the other hydrocarbons, particularly those from the first gas mixture (i.e. from the steam cracking process) are preferably not. As also explained in detail hereinafter, the second fraction is advantageously poor in hydrocarbons with one to three carbon atoms. It thus contains hydrocarbons with one to three carbon atoms in amounts of only up to 20%, 10%, 5%, 1%, 0.1%, 0.01% or 0.001% on a molar, weight or volume basis.

Within the scope of the present invention it is thus possible to carry out mild or even very mild cracking of the hydrocarbons which are particularly suitable for this, and which are found particularly in the second gas mixture, so as to obtain the particular advantages of mild or very mild cracking conditions as described hereinbefore. In particular, the second gas mixture contains a comparatively high proportion of butenes which can be reacted under the mild cracking conditions to produce the high-value product butadiene.

The invention makes it particularly easy to carry out this selective treatment of the hydrocarbons which are present particularly in the second gas mixture. Unlike in US 2013/0172627 A1, for example, there is no need for combined fractions to be laboriously separated from one another beforehand. No additional media such as for extractive distillation and no comparatively complex equipment are required for this purpose.

This separate treatment of at least two streams with different compositions makes it possible to carry out a more efficient treatment of products which are produced by an integrated apparatus (combined apparatus) as mentioned hereinbefore from a steam cracking process and an oxygenate-to-olefin process.

The cracking conditions to which the hydrocarbons contained in the second fraction and previously in the second gas mixture are subjected in the steam cracking process are preferably mild to very mild. This is possible because there are no disturbing components that react to form undesirable products in the steam cracking and thus might, for example, interfere with a subsequent separation or the steam cracking process itself.

If, for example, a fairly large quantity of diolefins are present in a cracking furnace feedstock which is subjected to mild cracking conditions, there may be an intensive formation of solids and polymerisation as well as an associated so-called fouling in the steam cracking process and in the subsequent working up. This is not the case within the scope of the present invention, as, for one thing, significantly fewer diolefins are present in the second gas mixture and hence in the second fraction, compared with the first gas mixture and the first fraction, because of the different reaction methods. Subsequent separation of diolefins is notably laborious and is possible only with great difficulty by distillation, for example.

The cracking conditions to which the hydrocarbons contained in the second fraction and, before that, in the second gas mixture are subjected in the steam cracking process mean that less than 91%, 90%, 89%, 88%, 87%, 85%, 80% or 75% and more than 50% or 60% of the n-butane present is reacted. For further features and advantages of mild cracking conditions, reference may be made to the definitions provided hereinbefore.

In the present case, it is particularly apposite to cite the cracking conditions relating to n-butane, as this is present in the second fraction, is easy to detect and its use as an indicator of cracking severity is accepted in the art (cf. for example the above-mentioned article “Ethylene” in Ullmann's Encyclopedia of Industrial Chemistry). The conversion is thus given in the present case for the key component normally used, even though this may be present in a small amount.

It has been found that any iso-butane which may also be present in the second fraction, as well as longer-chain branched compounds, can readily be included in the mild cracking. The advantages of the invention, namely the targeted formation of high-value products and the fact that complex separating units are not needed, and particularly the absence of diolefins, are clearly preponderant.

Hydrocarbons which are present in the first fraction or formed therefrom may also be subjected at least partly to the steam cracking process if the diolefins are removed. In contrast to the mild cracking conditions, however, severe or at least normal cracking conditions are preferably used to process these hydrocarbons.

Here, too, it has proved advantageous to select the cracking conditions according to the key component n-butane. Advantageous cracking conditions for processing the first fraction are, for example, those which result in more than 92%, 93%, 94% or 95% of the n-butane present being reacted.

Hydrocarbons which are formed from other hydrocarbons present in the first fraction may for example be compounds that have been hydrogenated or structurally changed in other ways by known methods. In other words it is possible to react the hydrocarbons contained in the first fraction in one or more steps, for example in order to arrive at compounds which can be particularly advantageously processed in a corresponding steam cracking process.

Ethane and propane from the first and/or second gas mixture may for example also be reacted by steam cracking in a so-called gas cracker, i.e. a cracking furnace designed for cracking C2 and C3 hydrocarbons. Once again, different cracking conditions may be used.

It is also possible to at least partially subject oxygenates contained in or formed from the second gas mixture and/or the second fraction to the oxygenate-to-olefin process. Corresponding oxygenates may also be recycled into the oxygenate-to-olefin process.

Moreover, it is also possible to at least partially subject hydrocarbons contained in the second gas mixture but not in the second fraction or formed therefrom to the steam cracking process. This may also be carried out under different cracking conditions.

The invention makes it possible, in the embodiments described, to conduct the process in a selective manner which may be adapted to the desired products and thus proves to be particularly flexible by comparison with known methods.

Within the scope of the method according to the invention, at least two or three cracking furnaces or furnace cells operated under different cracking conditions are advantageously used. At least one cracking furnace is provided which operates under the mild cracking conditions mentioned previously and which is charged with the above-mentioned second fraction, i.e. the fraction which contains the overwhelming majority of the hydrocarbons with four carbon atoms contained in the second gas mixture, and which comes from the oxygenate-to-olefin process.

As already mentioned, this first fraction is preferably poor in hydrocarbons with three or fewer carbon atoms. The second fraction is therefore formed from the second gas mixture, by separating off at least the great majority of the hydrocarbons with at most three carbon atoms which were previously contained in the second gas mixture, for example using a depropanizer or a corresponding separation sequence.

It is possible to combine the hydrocarbons having one, two and/or three carbon atoms, which have been separated from the second gas mixture, at least partly with the first gas mixture and/or with at least one fraction formed from the first gas mixture, to form at least one combined stream. Thus, in other words, after the hydrocarbons intended for mild cracking have been separated from the second gas mixture, the residue remaining can be combined with the first gas mixture or a corresponding fraction.

It is particularly advantageous to form at least two further fractions from the combined stream, i.e. to subject the combined stream to a joint separation. Thus, after the hydrocarbons of the second gas mixture intended for the mild cracking have been separated off, a simple joint separation can be carried out without wasteful use of resources.

The method according to the invention is also advantageous if the steam cracking method operates completely without fresh feed, i.e. only hydrocarbons contained in the first gas mixture and/or in the second gas mixture or formed therefrom are subjected to the steam cracking process. Such a process thus requires only an oxygenate feed, and there is no need to provide a separate fresh feed specially for the steam cracking process.

The present invention also relates to an apparatus for obtaining olefins. Such an apparatus comprises means that are designed to produce a first gas mixture by means of a steam cracking process and a second gas mixture by means of an oxygenate-to-olefin process, so that the first gas mixture and the second gas mixture each contain hydrocarbons with one to four carbon atoms.

According to the invention, means are further provided which are designed to form, from the first gas mixture, a first fraction which contains at least the great majority of the hydrocarbons with four carbon atoms previously contained in the first gas mixture, and to form, from the second gas mixture, a second fraction which contains at least the great majority of the hydrocarbons with four carbon atoms previously contained in the second gas mixture, and in the steam cracking process to subject the hydrocarbons contained in the second fraction and, previously, in the second gas mixture predominantly to cracking conditions in which any n-butane present is reacted by less than 92%.

An apparatus of this kind advantageously comprises all the means that are designed to perform the method, as explained hereinbefore. The apparatus therefore benefits from the advantages described hereinbefore, to which reference may expressly be made here. In particular, a corresponding apparatus comprises two or three cracking furnaces which are designed for operation under different cracking conditions.

The invention is described in more detail with reference to the attached FIGURE which shows a preferred embodiment of the invention.

### BRIEF DESCRIPTION OF THE DRAWING

FIG. 1 shows steps of a method for producing olefins according to one embodiment of the invention.

### EMBODIMENT OF THE INVENTION

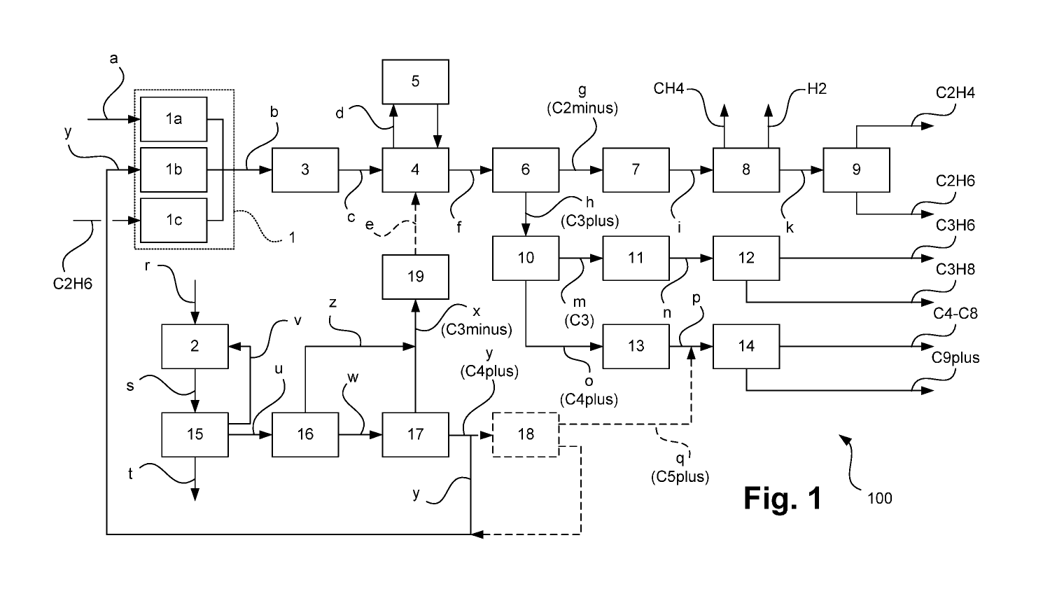

FIG. 1 shows a method according to one embodiment of the invention schematically in the form of a flow chart. The method as a whole is designated 100.

The method 100 comprises carrying out a steam cracking process 1 and an oxygenate-to-olefin process 2 in parallel. An apparatus in which the method 100 is implemented comprises corresponding means, i.e. in this case a plurality of cracking furnaces and at least one oxygenate-to-olefin reactor.

In the embodiment shown, the steam cracking process 1 operates using a plurality of feed streams which can be supplied to a plurality of cracking furnaces operated under different cracking conditions:

In the embodiment shown, three cracking furnaces 1a, 1b and 1c are shown. For example, the cracking furnace 1a is operated under severe or normal cracking conditions and a stream a, for example a fresh feedstock and/or a recycle stream is fed into this furnace. The stream illustrated as “a” may be formed from a plurality of streams. As already explained, the method according to the invention may also comprise the exclusive use of recycle streams in the steam cracking process 1. Recycle streams may be, for example, ethane and/or propane streams and/or streams of hydrocarbons with four to eight carbon atoms (olefinic and paraffinic). Fresh feedstocks may be supplied in gaseous and/or liquid form, for example in the form of natural gas and/or naphtha.

In the embodiment shown, the cracking furnace 1b is operated under mild cracking conditions and supplied with at least one stream y. The stream y is produced as a fraction of a gas mixture s (designated here as the second fraction or second gas mixture) formed by means of the oxygenate-to-olefin process 2. The stream y contains at least the hydrocarbons with four carbon atoms contained in the second gas mixture s, and optionally also longer-chained hydrocarbons (see below). The core of the present invention is the recycling of these and preferably only these hydrocarbons for mild cracking in the cracking furnace 1b.

Moreover, in the embodiment shown, another cracking furnace 1c is shown which is referred to as a so-called gas cracker and can be supplied with suitable feedstock streams such as ethane C2H6, as shown here. Other gaseous feeds are also suitable. The cracking furnace 1c can be operated under again different cracking conditions as compared to the cracking furnaces 1a and 1b.

Using the steam cracking process 1, over all a gas mixture b is obtained which is referred to here as the first gas mixture and which can be subjected to one or more preparation steps. In the embodiment shown, for example, an oil fractionation and/or a quenching are carried out in a step 3. Process steam may be produced which can be recycled into the steam cracking process 1 (not shown).

A gas stream c obtained in step 3 is subjected for example to compression, pre-cooling and drying in a step 4. Such a step 4 may also be supplemented by the elimination 5 of sour gas, with the formation of corresponding streams d. In the sour gas elimination 5 a gas stream is diverted off from step 4 between two compression stages into the sour gas elimination 5, for example, and fed back in later.

In the step 4, for example, a C3minus stream e which is formed from a corresponding second gas mixture s of an oxygenate-to-olefin process 2 can be used, as explained hereinafter. The result of the joint use of the stream c and the C3minus stream e from the oxygenate-to-olefin process 2 is that a corresponding pre-treatment only has to be carried out once and does not have to be done again separately for the comparatively small amounts of C3minus hydrocarbons from an oxygenate-to-olefin process 2. However, this is optional.

In the embodiment shown a stream f obtained from step 4, particularly one which has been compressed and partially liquefied and dried, is subjected, as a separation feedstock, to a deethanizer step 6 in which a C2minus fraction g and a C3plus fraction h is obtained. The further processing of the C3plus fraction h is explained hereinafter. The C2minus fraction g is subjected for example to a hydrogenation step 7 in which acetylene is hydrogenated to form ethylene, in particular.

A stream i further treated in this way is then subjected, for example, to a demethanizer step 8 in which methane CH4 and hydrogen H2 are separated off. A stream k thus freed from methane and hydrogen, which essentially still contains hydrocarbons with two carbon atoms, is subjected to a C2 separating step 9 (for example in a so-called C2 splitter) in which essentially ethylene C2H4 and ethane C2H6 are formed. The ethylene C2H4 is removed from the method 100 as a product, and the ethane C2H6 can be recycled into the steam cracking process 1, for example (see gas cracker 1c). If the process is designed accordingly, a method 100 according to the invention can also operate solely with recycled streams in the steam cracking process 1.

The C3plus stream h from the deethanizer step 6 is subjected to a depropanizer step 10. In the depropanizer step 10, a C3 fraction m is formed which can be worked up in one or more further process steps. For example, the C3 fraction m is subjected to a hydrogenation step 11 so that any methyl acetylene present as well as propadiene is reacted to form propylene. The stream thus processed, now designated n, is subjected for example to a C3 separating step 12 in which essentially propylene C3H6 and propane C3H8 are formed. The propylene C3H6, in turn, may be removed as product from a corresponding method 100 and the propane C3H8 may be recycled into the steam cracking process 1, for example into the gas cracker 1c.

A C4plus fraction o also formed in the depropanizer step 10 is subjected for example to full or partial hydrogenation in a hydrogenation step 13. A stream p obtained is fed into a deoctanizer step 14 in which essentially a C4 to C8 stream and a C9plus stream (without abbreviated names) are formed. If a corresponding stream is produced, a C5plus stream q which is produced from the second gas mixture formed in the oxygenate-to-olefin process 2, may also be fed into the deoctanizer step 14. The C9plus stream is removed from the process 100, whereas the C4 to C8 stream can be recycled back into the steam cracking process 1.

The oxygenate-to-olefin process 2 is particularly designed for reacting dimethyl ether, but methanol and other oxygenates, for example, and even partially or exclusively olefinic hydrocarbons may also be reacted. Corresponding oxygenates are supplied as stream r to one or more reactors and reacted to form a gas mixture s containing olefins, which is referred to here as the second gas mixture. The second gas mixture s, which contains at least or predominantly hydrocarbons with one to five carbon atoms, is subjected to an after-treatment step 15, for example water quenching and the elimination of oxygenates. Water obtained accordingly is drawn off as the stream t, and a stream u freed from oxygenates is fed into a step 16, which will be explained hereinafter. Any oxygenates recovered may be recycled as stream v into the oxygenate-to-olefin process 2.

In step 16, which has already been mentioned, the stream u is compressed and optionally pre-cooled. As already explained above, condensable components of the stream u are condensed. Any condensate obtained is optionally dried and subjected as a liquid stream w to a depropanizer step 17 in which a C3minus fraction x and a C4plus fraction y are formed from the stream w. The C4plus fraction y contains comparatively small amounts of hydrocarbons with five or more carbon atoms, for example, when corresponding catalysts are used (see above) in the oxygenate-to-olefin process 2. However, if necessary, a separating step 18 may be provided for the purpose of separating off corresponding longer-chained hydrocarbons as the stream q mentioned above. The latter can be further treated as mentioned above. The stream y, which is a C4plus or C4 stream, is recycled into the steam cracking process 1, as also mentioned previously, and cracked under mild conditions in the cracking furnace 1b.

Whereas the embodiment of the method according to the invention illustrated in FIG. 1 operates using a depropanizer 17, the invention may also be carried out in the same way using a separating sequence with a deethanizer. What matters is that a C4plus or C4 stream is formed from the second gas mixture or the stream s, recycled into the steam cracking process 1 and cracked under mild conditions therein.

The C3minus stream x may be combined with a stream z which consists of components that cannot be condensed in the condensation step 16, and subjected to an oxygenate removal step 19. An oxygenate stream (not shown) separated off in the oxygenate removal step 19 may be combined with the stream v and re-subjected to the oxygenate-to-olefin process 2. A C3minus stream freed from oxygenates, the stream e mentioned previously, may subsequently be subjected to the process step 4 described previously. However, this is optional.

1. Method (100) for obtaining olefins, wherein a first gas mixture (b) is produced by means of a steam cracking process (1) and a second gas mixture (s) is produced by means of an oxygenate-to-olefin process (2), the first gas mixture (b) and the second gas mixture (s) containing at least hydrocarbons with one to four carbon atoms,
characterised in that
from the first gas mixture (b) a first fraction (h) is formed which contains at least the great majority of the hydrocarbons with four carbon atoms previously contained in the first gas mixture (b),
from the second gas mixture (s) a second fraction (y) is formed which contains at least the great majority of the hydrocarbons with four carbon atoms previously contained in the second gas mixture (s), and
the hydrocarbons contained in the second fraction (y) and previously in the second gas mixture (s) are predominantly subjected in the steam cracking process (1) to cracking conditions under which any n-butane present is reacted by less than 92%.

2. Method (100) according to claim 1, wherein under the cracking conditions to which the hydrocarbons contained in the second fraction (y) and, before that, in the second gas mixture (s) are subjected in the steam cracking process (1), less than 91%, 90%, 89%, 88%, 87%, 85%, 80% or 75% and more than 50% or 60% of the n-butane present is reacted.

3. Method (100) according to claim 1 or 2, wherein any hydrocarbons which are present in the first fraction (h) or formed therefrom are also at least partly subjected to the steam cracking process (1).

4. Method (100) according to one of the preceding claims, wherein oxygenates contained in the second gas mixture (s) or formed therefrom are at least partially subjected to the oxygenate-to-olefin process (1).

5. Method (100) according to one of the preceding claims, wherein hydrocarbons previously contained in the second gas mixture (s) but not in the second fraction (y), or formed therefrom, are at least partially subjected to the steam cracking process (1).

6. Method (100) according to one of the preceding claims, wherein the steam cracking process is carried out using at least two cracking furnaces (1a-1c) operated under different cracking conditions.

7. Method (100) according to one of the preceding claims, wherein the oxygenate-to-olefin process (2) is partially or exclusively carried out using at least one olefin which is, in particular, not present in the first and/or second gas mixture (b, s).

8. Method (100) according to claim 7, wherein the oxygenate-to-olefin process (2) is carried out using at least two reactors which are operated under different conditions.

9. Method (100) according to one of the preceding claims, wherein the second fraction (y) is formed from the second gas mixture (b) such that it is poor in hydrocarbons having one to three carbon atoms.

10. Method (100) according to claim 9, wherein the second fraction (y) is formed from the second gas mixture (s) by separation of at least the great majority of the hydrocarbons with at most three carbon atoms that were previously contained in the second gas mixture (s).

11. Method (100) according to claim 10, wherein hydrocarbons having one, two and/or three carbon atoms, separated from the second gas mixture (s), are combined at least partially with the first gas mixture (b) and/or with at least one fraction formed from the first gas mixture (b) to form at least one combined stream.

12. Method according to claim 11, wherein at least two further fractions are formed from the combined stream.

13. Method according to one of the preceding claims, wherein only hydrocarbons contained in the first gas mixture (b) and/or in the second gas mixture (s) or formed therefrom are subjected to the steam cracking process (1).

14. Apparatus for obtaining olefins, which comprises means that are designed to produce a first gas mixture (b) by means of a steam cracking process (1) and a second gas mixture (s) by means of an oxygenate-to-olefin process (2), so that the first gas mixture (b) and the second gas mixture (s) at least contain hydrocarbons with one to four carbon atoms,
characterised by means which are designed
to form, from the first gas mixture (b), a first fraction (h) which contains at least the great majority of the hydrocarbons with four carbon atoms previously contained in the first gas mixture (b),
to form, from the second gas mixture (s), a second fraction (y) which contains at least the great majority of the hydrocarbons with four carbon atoms previously contained in the second gas mixture (s), and
in the steam cracking process, to subject the hydrocarbons contained in the second fraction (y) and, previously, in the second gas mixture (s) predominantly to cracking conditions under which any n-butane present is reacted by less than 92%.

15. Apparatus according to claim 14, which comprises means designed to perform a process according to one of claims 1 to 13.

16. Apparatus according to claim 14 or 15, which comprises at least two cracking furnaces (1a-1c) which are designed for operation under different cracking conditions.
